(19) United States
(12) Patent Application Publication
(10) Pub. No.: US 2025/0084362 A1
(43) Pub. Date: Mar. 13, 2025

(54) SYSTEM AND METHOD FOR CELL PROCESSING

(71) Applicant: CELLUNITE GMBH, Munich (DE)

(72) Inventor: Armin KESHMIRI, Munich (DE)

(73) Assignee: CELLUNITE GMBH, Munich (DE)

(21) Appl. No.: 18/580,764

(22) PCT Filed: Jul. 20, 2022
(86) PCT No.: PCT/EP2022/070400

§ 371 Date: Jan. 19, 2024

(30) Foreign Application Priority Data
Jul. 23, 2021 (EP) 21187485.4

(51) Int. Cl. C12M 1/36; C12M 1/00; C12M 1/26; C12M 3/00

(52) U.S. Cl. CPC C12M 41/48; C12M 23/44; C12M 33/04; C12M 45/05; C12M 45/20

(57) ABSTRACT

The present invention is directed to a system, method, use, and diagnostic kit for cell-derived processing. The invention comprises a controlling component and a driving and/or heating component configured to host a cartridge and drive and/or heat the cartridge.

### FIELD

The present invention relates to a system and a method for an automated tissue processing. The automated and/or semi-automated tissue processing can be controlled automatically.

### INTRODUCTION

The current state of the art includes various mechanical and enzymatic processes for the production of stem cell products. One is mechanical-processing: centrifuges are used for the preparation of blood and tissue products, as well for analysis or autologous therapy applications. For example, there are methods such as platelet rich plasma (PRP), fat processing, and bone marrow processing in centrifuges for the generation of a stem cell suspension (SVF). The other option is manual centrifuges.

During the centrifugation of the fat cell extract, various sources add enzymes, such as conventional collagenases, to free the fat extract from the fat cells and extracellular matrix in order to obtain a purer stem cell suspension.

Machines for automated fat tissue and bone marrow processing are also known. These are equipped for centrifugation and addition of enzymes, in a closed manner. Some of them are also configured to add enzymes by manual shaking and then performing the automated centrifugation.

US 2004/0106196 A1 is directed to Methods of treating patients include processing adipose tissue to deliver a concentrated amount of stem cells obtained from the adipose tissue to a patient. The methods may be practiced in a closed system so that the stem cells are not exposed to an external environment prior to being administered to a patient. Compositions that are administered to a patient include a mixture of adipose tissue and stem cells so that the composition has a higher concentration of stem cells than when the adipose tissue was removed from the patient.

In US 2005/0260175 A1 systems and methods are described that are used to separate cells from a wide variety of tissues. In particular, automated systems and methods are described that separate regenerative cells, e.g., stem and/or progenitor cells, from adipose tissue. Tue systems and methods described herein provide rapid and reliable methods of separating and concentrating regenerative cells suitable for re-infusion into a subject.

US 2020/0032197 A1 concerns a single use aseptic kit comprising: a disaggregation module for receipt and processing of material comprising solid mammalian tissue; and a stabilization module for storing disaggregated product material, wherein each of the modules comprises one or more flexible containers connected by one or more conduits adapted to enable flow of the tissue material there between; and wherein each of the modules comprises one or more ports to permit aseptic input of media and/or reagents into the one or more flexible containers. The invention further relates to an automated device for semi-automated aseptic disaggregation and/or enrichment and/or stabilization of cells or cell aggregates from mammalian solid tissue comprising a programmable processor and the single use aseptic kit. The invention further relates to a semi-automatic aseptic tissue processing method.

WO 2019/145029 A1 relates to a device and a method for dissociating tissue. In one embodiment, the device comprises at least one dissociation unit for receiving a tissue sample, which is arranged at least partially in a pot-shaped cell sieve. The invention also relates in an embodiment to a device and a method for dissociating tissue, wherein the tissue is dissociated by means of a grinding mechanism having rows of teeth which can be moved relative to one another and which, when moving past one another in the one direction, cut the tissue and, when moving past one another in the opposite direction, grind the tissue.

For example, U.S. Pat. No. 9,144,583 B2 provides automated devices for use in supporting various cell therapies and tissue engineering methods. An automated cell separation apparatus is capable of separating cells from a tissue sample for use in cell therapies and/or tissue engineering. The cell separation apparatus can be used in combination with complementary devices such as cell collection device and/or a sodding apparatus to support various therapies. The automated apparatus includes media and tissue dissociating chemical reservoirs, filters, a cell separator and a perfusion flow loop through a graft chamber which supports a graft substrate or other endovascular device. The present invention further provides methods for using the tissue grafts and cell samples prepared by the devices described herein in a multitude of therapies including revascularization, regeneration and reconstruction of tissues and organs, as well as treatment and prevention of diseases.

Further, EP 2714887 A1 provides an automated system for isolating stromal vascular fraction cells from the mammalian tissue. The system comprises a plurality of containers for storing buffer solutions, tissue samples and digestive buffers. A tissue processing unit fluidly connected to the containers for processing the tissues. The tissue processing unit performs at least one of a washing processes, digestion process, phase separation process and combination thereof for separating an aqueous fraction of tissue and a fatty fraction. A cell concentration unit fluidly connected to the tissue processing unit for receiving the aqueous fraction of tissue from the tissue processing unit. The cell concentration unit filters the aqueous fraction of tissue by vibrating a filtration assembly by a filter vibrator. A waste collection unit fluidly connectable to the tissue processing unit and cell concentration unit is provided for receiving waste tissues. The system further comprises a control unit to control the operation of the system.

Also, U.S. Pat. No. 7,390,484 B2 is directed to cells present in processed lipoaspirate tissue that is used to treat patients. Methods of treating patients include processing adipose tissue to deliver a concentrated amount of stem cells obtained from the adipose tissue to a patient. The methods may be practiced in a closed system so that the stem cells are not exposed to an external environment prior to being administered to a patient. Compositions that are administered to a patient include a mixture of adipose tissue and stem cells so that the composition has a higher concentration of stem cells than when the adipose tissue was removed from the patient.

Additionally, CN 109082408 A discloses a device for fragmenting fat tissue, described device includes two no less than two syringes and connecting tubes, and for syringe by nipple and connecting tube connection, the connecting tube has a no less than bending structure, connecting tube minimum diameter 3 mm. After adipose tissue is crushed using device provided by the invention, the time of digestion fat stem cell and the dosage of clostridiopeptidase A can significantly be reduced, only need to reach for 20 minutes good digestion effect, while the quantity of the stem cell obtained is 4 times of conventional method or more. It is repeatedly centrifuged the clostridiopeptidase A for effectively eliminating digestion simultaneously, ensure that the safety for the fat stem cell being separated to, there is the value and potentiality of very big popularization and application.

Further, CN 107224614 A discloses a kind of preparation and its clinical practice of the adipose-derived stromal cells co-graft material rich in cell factor. Acquisition and purifying that preparation method is rolled into a ball by 1) lipochondrion; 2) acquisition of nanometer adipose stromal cells; 3) biological characteristics of nanometer adipose-derived stem cells (NFSCs) and multidirectional differentiation identification; 4) four steps of acquisition rich in cell factor fibrin glue prepare graft materials, reparation and correction for clinical upper surface portion, chest and other body surface soft tissue depressions deformity. The technology described promotes wound or wound repair based on stromal vascular fraction and reduces the new technology that scar proliferation, promotion organization regeneration and repair and organ are rebuild, shorten the rehabilitation duration of patient, improve quality of rehabilitation, good curative effect is obtained, the autologous adipose tissue skin grafting and mending soft tissue or organ defect, burn wound, refractory wounds and mammary cancer breast reconstruction aided in for clinically cell provides experimental basis and theories integration.

Also, KR 101489264 B1 relates to a stem cell isolation kit and a stem cell isolation method for separating stem cells from various tissues of the human body. The stem cell separation kit comprises: an upper body having an upper accommodation space and a lower accommodation space; and a second accommodation space having a conical shape at its lower end with a cross section smaller than the first accommodation space and having a cone shape; a lower main body; a separating bar capable of being raised and lowered through the upper main body and closing the upper end of the neck portion when the lower portion is lowered to block fluid communication between the upper accommodating space and the lower accommodating space; a squeezing network detachably mounted in the first receiving space of the closed lower body; and the material accommodated in the upper accommodation space may be filtered through the sieve filter to separate a substance having a predetermined size or larger. According to such a configuration, since the separated stem cells are isolated from one container to another, and a single stem cell separation kit is used to perform operations such as separation, washing and separation of stem cells, A stem cell separation kit and a stem cell separation method in which a separation operation can be performed.

KR 101893819 B1 relates to a method for quality control of stem cells using electrophoresis and cell migration assays. More particularly, the present invention relates to a method for quality control of stem cells by distinguishing stem cells from undifferentiated stem cells and bone differentiation by applying a DC electric field to a stem cell sample, and a quality control apparatus using the same. By using the method according to the present invention, it is possible to easily determine the undifferentiated stem cells and the differentiated stem cells by applying an electric field to the stem cell samples, thereby judging the quality of the stem cell samples. In particular, by applying an electric field formed by DC to an adipose-derived stem cell sample, it is possible to easily and effectively discriminate undifferentiated adipose-derived stem cells and osteocyte-differentiated stem cells.

U.S. Pat. No. 9,956,317 B2 describes methods and kits for producing cellular fractions enriched in adipose derived stem cells. Methods are provided where adipose tissue obtained from liposuction is enzymatically treated using a solution containing collagenase and divalent cations prior to the application of traditional methods of stromal-vascular fraction isolation. The enzymatic solutions may contain collagenase types I and II to a final concentration of about 0.001 mg/ml to 0.010 mg/ml. The divalent cations may be present as calcium, magnesium, and zinc chloride. The final concentration of calcium, magnesium, and zinc may range from about 0.001 to 0.1 micromolar; about 0.005 to 0.5 micromolar; and about 0.0015 to 0.15 micromolar, respectively. The enzymatic solutions may be generated using a kit where the collagenase and divalent components are held in separate containers until just prior to use. The cellular fractions isolated in this manner may be used in autologous fat grafts in therapeutic applications.

WO 2017078563 A1 relates to medical biotechnology and cell technology. Proposed is a method for isolating stromal vascular fraction of adipose tissue, which includes: decantation of a lipoaspirate; delicate washing of the lipoaspirate in buffer solutions; enzymatic treatment using a mixture of collagenases, thermolysis, dispose and trypsin at a temperature of 37° C.; centrifugation at 500-2000 g and filtration through microfilters with a pore size of 10-300 microns with the subsequent removal of fat and stromal tissue matter; repeat washing, and concentration of the cell fraction, wherein all of the stages of the process are carried out under closed system conditions. A corresponding device is configured as a sealed container consisting of two chambers, which are separated from one another by nylon microfilters with pore sizes of 10-500 microns. The device comprises fittings, provided with valves, and channels for the introduction and removal of biological tissue and a cellular end product for subsequent administration. Owing to the presence of a cocktail of mature and progenitor cells in stromal vascular fraction, which have a paracrine effect, the sterility of all of the stages of the process and the safety thereof, the claimed method can be used in medicine for the cell-based therapy and regeneration of organs and tissues, including: soft tissue and bone regeneration, and the treatment of cosmetic defects, chronic trophic and radiation ulcers, burns, Crohn's disease, multiple sclerosis, graft-versus-host reactions, myocardial infarction and strokes of different origins.

AU 2002357135 B2 is directed to a self-contained adipose-derived stem cell processing unit, comprising: a tissue collection container that is configured to receive unprocessed adipose o s tissue that is removed from a patient, wherein said tissue collection container is defined C s by a closed system; a first filter that is disposed within said tissue collection container, wherein said n first filter is configured to retain a first component of said unprocessed adipose tissue and pass a second component of said unprocessed adipose tissue, such that said first filter I separates said first component from said second component, and wherein said first C to component comprises a cell population that comprises adipose-derived stem cells and said second component comprises lipid, blood, mature adipocytes, and saline; a cell collection container, which is configured to receive said first component comprising a cell population that comprises adipose-derived stem cells from said tissue collection container, wherein said cell collection container is within said closed system; a conduit configured to allow passage of said first component comprising a cell population comprising adipose-derived stem cells from said tissue collection container to said cell collection container while maintaining a closed system; a cell concentrator disposed within said cell collection container, which is configured to facilitate the concentration of said first component comprising a cell population that comprises adipose-derived stem cells so as to obtain a concentrated population of cells that comprise adipose-derived stem cells, wherein said cell concentrator comprises a centrifuge or a spinning membrane filter; and an outlet configured to allow the aseptic removal of said concentrated population of cells that comprise adipose-derived stem cells.

DE 102013209718 B4 relates to a device for separating adult stem cells from an adipose tissue taken from a biological structure. The device has a container for receiving a substance mixture which comprises the fatty tissue and the adult stem cells. Furthermore, the device has a flushing agent supply device, a substance mixture supply device, a stem cell removal device, a flushing agent removal device and a specifically permeable membrane. In this case, the container has at least two chambers, which are separated by the at least one membrane. The substance mixture supply device and the stem cell removal device are also separated from one another by the at least one membrane.

IT GE20120034 A1 relates to the preparation and method for producing a preparation or a tissue derivative comprising mesenchymal stem cells, to be used in cellular therapy, for cosmetic treatments, for replacing a tissue or an organ, or inducing or accelerating tissue repair or regeneration. Said method provides at least the following steps: extraction of tissue containing mesenchymal stem cells, such as adipose tissue, from a cadaveric donor by liposuction process or by surgical removal of parts of adipose tissue, mechanical treatment of said tissue, said tissue, such as the adipose tissue, being composed of a fluid component comprising an oily component, a blood component and/or sterile solutions and of a solid component comprising cell fragments, cells and one or more cell macro-agglomerates of heterogeneous sizes, and said mechanical treatment step being provided for separating and removing the fluid component from the solid component, which step of mechanical treatment separating and removing the fluid component from the solid component provides an emulsion of fluid components to be generated, by mechanical stirring.

The aforementioned described processes are often cumbersome and time-consuming and result in an unsatisfying outcome. Further, the existing processes provides a low cell viability.

### SUMMARY

In light of the above, it is an object of the present invention to overcome or at least alleviate the shortcomings of the prior art. More particularly, it is an object of the present invention to provide a method and a corresponding system for tissue derived cell processing is disclosed.

The present invention is directed to a system and a method. Wherever one of those categories and its features are discussed the same applies for the other category and its features mutatis mutandis.

The present invention relates to a system and method for tissue derived cell processing comprising a controlling component. The controlling component can host the electronic components, such as a CPU, a storage, interfaces etc. that can constitute the core of the system. It can further comprise a driving and/or heating component configured to host a cartridge and to drive and/or heat a cartridge. Both, the controlling component and the driving and/or heating component can be provided integrally, remotely to each other or can be provided in a modular combinable way.

A cartridge can comprise a shaker and/or a centrifuge. The cartridge can be configured to be used in a system according to the present specification.

It can particularly comprise a shaker and/or a centrifuge for shaking and/or centrifuging lipoaspirate. The lipoaspirate can be the material aspirated from a patient to be then further processed by the present invention to particularly obtain a cell- or stem cell-enriched substance. A shaker and a centrifuge can be hosted in the cartridge. The shaker can host a sieve that is locate in the cartridge. The sieve can comprise one or more variable mesh size that can be particularly realized by at least two mesh layers that can be moved to each other, such as rotated to each other.

The cartridge can comprise an input and/or an output that are configured to be fluidly connected to another component, such as the driving and/or heating component and/or the controlling component.

The output can comprise an outlet for a cell-enriched substance and a second outlet for a cell-impoverished substance. The first and second outlets can be concentrically arranged. The first and second outlets can be concentrically arranged with the outlet for the cell-enriched substance being located exterior to the second outlet for the cell-impoverished substance.

The shaker can be configured to also shake the centrifuge.

The cartridge can be connected by and driven and/or heated by the driving and/or heating component.

The controlling component and the driving and/or heating component can be modular and connected to each other so that the controlling component can control the driving and/or heating component.

The controlling component can comprise at least one slot for the insertion of a reservoir for a substance.

The controlling component can comprise at least one slot for the insertion of a syringe of a substance and wherein the controlling component can comprise an expelling component for expelling the content of the syringe(s) in controlled fashion.

The expelling component can comprise one or more activator(s) for a/each plunger of the syringe(s) wherein each activator is able to push and/or retract the plunger of the syringe(s).

The controlling component and the driving and/or heating component can be configured to be connected to each other with a liquid connection in order to enable any substance to be transported to and/or through the driving and/or heating component.

The driving and/or heating component and the cartridge can be configured to be connected to each other with a liquid connection in order to enable any substance to be transported to and/or through the driving and/or heating component into and/or out of the cartridge.

The system can further comprise one or more valves configured to enable a flow of fluids from the controlling component to the cartridge and/or vice versa. The valves can be configured to enable a flow of fluids from the controlling component to the cartridge and/or vice versa.

There can be provided one or more valves configured to enable a flow of fluids from the controlling component to the cartridge and/or vice versa through the driving and/or heating component. All kinds of valves can be used, such as stop valves and 2-way as well as 3-way and other valves.

The controlling component and the driving and/or heating element can be configured to be electrically and/or fluidly connected. They can also be configured as a snap-in or click-in connection.

The controlling component and the driving and/or heating element can be configured for a connection verification upon a proper connection. This verification can be a mechanical and/or electronical element, such as a flag or identification of any kind. It can alternatively or additionally be displayed on a user's interface, such as on a GUI. A connection locking, such as a self-locking element can be provided so that once a safe lock is identified the components cannot be unintentionally unlocked. A lever or software lever can unlock again.

The driving and/or heating element and the cartridge can be configured to be electrically and/or fluidly connected. In case of both, two respective interfaces, plugs and/or locks etc. can be provided.

The driving and/or heating element and the cartridge can be configured for a snap-in connection. The connection verification upon a proper connection can be realized. A male member for a connection between both.

The controlling component can comprise a user interface, such as a GUI. The controlling component can comprise a surface with a user interface and/or syringe slots. The surface can slope towards the front towards a potential user. The controlling component can be also configured to host a container for cell enriched substance. This can be a product recess for a container with cell enriched substance, such as a product recess for a syringe with cell enriched substance.

The system can also comprise a cell quantifying and/or qualifying component which may be configured for quantifying the cells in the derived tissue. The term cell is understood to also comprise regenerative cells and/or any vesicles and/or parts thereof, such as exosomes/surface proteins. The cell quantifying and/or qualifying component can be configured for quantifying the volume flow in the system. This can also comprise the qualifying the flow in the system.

The volume flow of the system can be defined as being one or more of the following: cell number, cell quality, cell components, vesicle and/or parts thereof, such as exosomes/surface proteins, advantageously obtained by impedance measurement, cell mass flow and/or cell volume flow, cell quality etc. advantageously obtained by optical measurement, volume flow, mass flow and/or density flow, advantageously obtained by an optical sensor, volume, weight, density and/or viscosity of the tissue.

In some embodiments the tissue may comprise an adipose tissue, bone marrow tissue or the alike. In some further embodiments the cells can comprise stem cells derived from the adipose tissue. The cells can also comprise a composition of stem cells. Further, the cells can comprise progenitor cells, pericytes. The cells can be pericytes and/or cytokines. The cell composition may be cells containing proteins, etc.

In some embodiments the system comprises an AMFAT component. The AMFAT component can be receiving an input via an inlet from a cell quantifying and/or qualifying component. In such embodiments the cell quantifying and/or qualifying component may be configured to measure quantity and/or quality of the tissue.

The cell quality may be defined by the quantity of undifferentiated stem cells and/or the quantity of differentiated stem cells and/or the ratio of undifferentiated stem cells versus differentiated stem cells present in the tissue. In particular, the undifferentiated stem cells can be adipose-derived and the differentiated stem cells can be cartilage or osteocyte-differentiated. Furthermore, the quality of the tissue can be derived from a cellularity measurement with antibodies, optically or with fluidic and/or microfluidic.

In some embodiments the system may comprise a centrifuge component. The centrifuge component can be configured to receive an input from at least one of the AMFAT component, cell quantifying and/or qualifying component.

In some embodiments the cell quantifying and/or qualifying component may be measuring tissues using impedance and/or DC resistance and/or sound propagation to measure the velocity of the volume flow. In such embodiments the cell quantifying and/or qualifying component can comprise impedance sensor and/or optical sensor and/or resistance sensor and/or sound source. In some embodiments the volume flow is first passed through the cell quantifying and/or qualifying component before the AMFAT component and/or the centrifuge component.

In some embodiments the cell quantifying and/or qualifying component can be configured to measure volume, weight, density, viscosity of the tissue. The system may further comprise at least one immunoassay unit and/or a colorimetric system and/or fluorescence measurement unit to quantify the antigens present in the tissue. In such embodiments the immunoassay unit and/or the colorimetric system and/or fluorescence measurement unit can be installed at the cell quantifying and/or qualifying component. The quantification can be via antigens present on the cell surface. In some embodiments the system may comprise more than one fluorescence measurement unit, wherein each fluorescence measurement unit may be configured to quantify more than one antigen. In some further embodiments the system may comprise more than one colorimetric system, wherein each colorimetric system may be configured to quantify more than one antigen. The system may further comprise a microfluid component. The microfluid component can be configured to quantify at least one antigen on the at least one microfluid component. In some embodiments the microfluid component can be configured to quantify a plurality of antigen on one microfluid component, such as a microfluid plate. In such embodiments the biproduct can remain in the microfluid component.

In a further embodiment the system can further be configured to generate cell-relevant data. The cell relevant data can comprise the data generated by the system after the at least quantification and/or qualification of the cell. The cell relevant data can further be generated by microfluid component and/or optical sensor and/or impedance sensor and/or resistance sensor and/or sound source and/or immunoassay unit and/or colorimetric system and/or fluorescence measurement unit.

In some embodiments the cell relevant data can comprise quantity of the cell and/or of the tissue which is inputted in the system. The quantity may comprise, mass, density, volume, viscosity. The cell relevant data can further comprise temperature of the volume flow in the system and/or the temperature of the input and/or the temperature of the output. In a further embodiment the cell relevant data can comprise enzyme data. The enzyme data can comprise a binary data for if an enzymatic digestion is required for the cell processing or not. In some embodiments the enzyme data can further comprise type of the enzyme needed for enzymatic digestion.

The following markers can be used, e.g. in an analysis component: CD217 for detection and/or quantification of mesenchymal stems and/or progenitor cells; CD146+ for the progenitor cells and chondrogenic progenitor cells; CD166+ for pericytes and/or chondrogenic progenitor cells; C13+; CD166 for mesenchymal stem and/or progenitor cells and/or chondrogenic progenitor cells, adipose-derived stem cells and chondrogenesis; CD105 for adipose-derived stem cells and chondrogenesis potential; CD90 for adipose-derived stem cells and/or CD271 for chondrogenesis potential and/or mesenchymal stem cells.

In some embodiments the cell quantifying and/or qualifying component can be configured quantify the at least one of the input sample and the end product. In some further embodiments the cell relevant data can comprise the input data and/or data quantified at an intermediate processing and/or cell relevant data quantified using the end product.

In some further embodiments the cell relevant data can comprise type of cell, such as cell types, metabolic activity, cell states etc. The cell relevant data can further comprise data related to additive. Additive can comprise the information about a component which can be added to generate an optimum end product. The additive can be hyaluronic acid and/or PRP.

In some further embodiments the cell relevant data can further comprise viscosity data, wherein the viscosity can be measure by a syringe derive. The viscosity can be measure by the syringe derive via force measurement. The syringe derive may comprise a syringe pump ad/or peristaltic pump and/or roller pump.

In some embodiments the cell relevant data can further comprise composition data, such as composition of the input volume flow. The composition can comprise a percentage concentration of cells in the input volume flow.

In some embodiments the cell relevant data may further comprise size data. The size data may comprise the size of fat clusters in the volume flow and/or the input tissue.

In some embodiments the AMFAT component and the cell quantifying and/or qualifying component can be integrated on a single device. In some further embodiments the immunoassay unit and the colorimetric system or the fluorescence measurement unit may be integrated on the same device. The cell processing component and/or the AMFAT component may further comprise a computing unit. The computing unit may also be configured to exchange data between the at least two components.

In a further embodiment the syringe driver can comprise a vacuum pump, which can be installed at an outlet of the microfluid component.

In a further embodiment the cell relevant data can be transferred to a server. The server may be a remote server and/or a local server. The cell relevant data can be encrypted first before sending it to the server. In such embodiments processing parameters such as swivel amplitude, centrifugation speed, enzyme quantity, temperature can be based on cell relevant data. In such embodiments a first processing can be performed with a pre-defined default parameter and the processing parameters can be generated based on the first processing results.

In some further embodiments wherein, the system can generate a vibration amplitude based on the cell relevant data. The swivel amplitude can then be used to configured a swivel component. The swivel component can be installed in the AMFAT component. The swivel component can comprise a shaker and/or a vibrator. The swivel component can further comprise a heat controller, an inlet and an outlet. The swivel component can be configured to shake tissues and hence perform washing/cleaning of the cell composition. The swivel component can comprise a swivel frequency in a range of 8 Hz to 100 Hz, such as 20 Hz to 80 Hz. In some further embodiments the swivel component can further be configured to provide a swivel amplitude in a range of 0.5 mm to 50 mm, such as 20 mm to 40 mm.

In some further embodiments the swivel component can further be configured with a waste unit. The waste unit can be configured to collect at least one component from the washing/cleaning in the swivel component. In some embodiments the at most volume of waste can be 600 ml. In a furthermore embodiment the swivel component can comprise a grid and/or a net and/or a mesh which can be configured to collect at least one waste particle.

The swivel component can be further configured with a computing unit which can be configured for the operation of the swivel component. The computing unit can further be configured to pull the operational parameters of the swivel component from the AMFAT component and/or the cell quantifying and/or qualifying component. In some embodiments the computing unit can set pre-defined parameters for the operations. In a further embodiment a user device can input the parameters.

In a further embodiment the AMFAT component can comprise an enzyme inlet. In such embodiments an enzyme component can be configured to introduce at least one enzyme in the AMFAT component via the enzyme inlet. The enzyme component can further be controlled by the computing unit which can control an amount of enzyme added based on the cell relevant data.

In some embodiments the system may comprise at least three syringe drivers. The one syringe driver may be used to input an enzyme to the swivel component and/or centrifuge component. At least one syringe driver can be used to input tissues in the system. The at least one syringe driver may be used to extract an end product from the system. The syringe driver can be configured to have a volume capacity in a range of 2 ml to 100 ml. The at least one syringe driver can be used to add at least one agent to an end product of the system. The at least one syringe driver, such as a roller pump can be used to pump sterile isotonic NaCl solution from the salinity component into the AMFAT component. In such embodiments the roller pump may comprise a volume capacity of at most 1000 ml.

In some embodiments the volume flow may be transferred from the swivel component to the centrifuge component. In such embodiments the at least one syringe driver may be used to facilitate this transfer. The speed of the transfer can comprise a range of 0.1 ml/s to 10 ml/sec, such that 1 ml/sec. The system may further be configured to pull syringe driver parameters and store them into the at least one of cell quantifying and/or qualifying component and server. For example, for each syringe driver how much force is needed to press a 50 ml syringe driver with grease into a tube with a syringe pump at a defined speed is pre-determined.

In some embodiments the swivel component may comprise a weight measuring component. The weight measuring component can comprise a load cell, which can be configured to measure a weight at a pre-determined interval of the content present in the weight measuring component.

In some embodiments the swivel component can be configured to be a part of the AMFAT component. Furthermore, the AMFAT component can comprise the cell quantifying and/or qualifying component. In some embodiments the AMFAT component may be configured to generate at least a portion of cell-relevant data. Such as, weight of the volume flow.

In some embodiments the cell relevant data may be generated by the swivel component after the shaking/vibrating/swivelling. This can be particularly advantageous to determine the effect of shaking on the tissue.

In some embodiments the syringe driver may be configured to input a portion of the volume flow in the cell processing component to generate at least a portion of cell relevant data. For example, in a first test run at most 20 ml of tissues can be used to generate the cell relevant data and hence determining the operational parameters. For example:
1. If the density of the supplied tissue is lower, a centrifuge speed of 2500 rpm can be set instead of 7000 rpm.
2. If the amount of added tissue is lower than previously specified, the amount of added enzyme should be reduced proportionally.

In some embodiments the AMFAT component can be equipped with a temperature controller. The temperature controller can have a pre-determined temperature setting. In some embodiments the optimum temperature can be determined based on the cell relevant data. The temperature controller can be configured to maintain an optimized temperature of the volume flow in the system and/or in the AMFAT component. In some embodiments the optimized temperature can be automatically determined by the cell quantifying and/or qualifying component based on cell-relevant data.

In some further embodiments the system can comprises additive component. The additive component can comprise the at least one syringe driver. The additive component can be configured to add at least one pre-determined additive to an end product. The additive to be added can be determined on the basis of cell relevant data and/or user data. The additives can comprise hyaluronic acid and/or platelet rich plasma (PRP).

In some embodiments the saline component may be configured to control the salinity of the volume flow in the system.

In some embodiments the system comprises the centrifugation component. The volume flow can automatically be transferred to the centrifuge component, preferably via the syringe driver. In some embodiments they system may further comprise a tube system, wherein the tube system can be configured to facilitate the volume flow in the system.

In some embodiments the centrifuge component may comprise a computing unit configured to control the operation parameters of the centrifuge component. The operational parameters of the centrifuge component can comprise centrifugation speed, centrifugation time, number of centrifugation cycles, temperature of the content.

In some embodiments the centrifuge component may comprise the weight measuring component. In some further embodiments the centrifuge component can further comprise the temperature controller. It may be noted in such embodiments the system may comprise a plurality of temperature controller and/or weight measuring component and/or computing unit.

In some embodiments the AMFAT component and the centrifuge component can be integrated on a single device.

In some embodiments the operational parameters of the centrifuge component are determined by the computing unit based on the cell relevant data. In some further embodiments the centrifuge component can comprise an inlet and/or an outlet. The inlet and/or the outlet may be connected to the syringe driver and/or the tube system.

In some further embodiments the system may comprise a user interface. The user interface may be integrated on the same device as the AMFAT component and/or the centrifuge component. The user interface may be configured to extract the user data which can then be added to the cell relevant data. The user data may comprise, age, gender, region, etc. The user data may further be automatically pulled by a user device, such as wearables, etc. The user data can then be communicated to the cell quantified component.

In some embodiments the user interface may comprise an input interface and/or an output interface. The output interface may comprise a screen configured to display at least one of processing time and processing step and components available (such as enzyme component, swivel component, centrifuge component etc.) and error and quantified results and detection of an external source, such as USB. The input may comprise a keyboard, joystick, touch screen to enter age, gender, etc. The input may further pull data related to confirmation of the insertion of syringe drivers. The input may further receive instructions for starting the cell processing and/or permission for a test run. The test run may comprise using a portion of the sample.

In some embodiments the system may be configured to be manually receiving operational parameters via the input interface of the user interface. The operational parameters may comprise centrifuge speed, swivel frequency etc. In some embodiments the user can further chose an enzyme addition via the user interface.

In a further embodiment the input interface may further be configured to communicate with the additive component based on the user input. For example, input interface may have following input from the user:
1. no enzyme, centrifuge fixed 5000 rpm, add hyaluronic acid,
2. with enzyme (automatic amount), centrifuge automatic speed depending on the measured values, hyaluronic acid is not added

In some further embodiments the user interface may be configured to stop the processing manually at any step.

In some embodiments the available measured values (cell relevant data) can be displayed to the user interface; the input interface can then decide whether hyaluronic acid/PRP or the enzyme should be added additionally.

In a second embodiment a method for cell processing is disclosed. The system is configured to perform any of the step according to the method.

In a third embodiment a use of the system to carry out any of the method steps is disclosed.

In a fourth embodiment a diagnostic kit is disclosed. The kit can comprise a disposable click-in kit. The kit can further be attached to the system. The kit can comprise the swivel component and/or the cell quantifying and/or qualifying component and/or the centrifuge component. The diagnostic kit can comprise a maximum weight of 30 kg. The diagnostic kit can comprise a size of approximately 200×100×70 cm, such that 120×70×50 cm.

In some embodiments the viscosity of the volume flow in the diagnostic kit can be in the range of 1 to 10 cP, such that 2 to 8 cP, more preferably 3 to 7 cP. The volume of the volume flow in the diagnostic kit can comprise a range of 40 to 600 ml, such that 50 to 500 ml.

The invention bears the further preferred advantages:

The modularity and feed-back approach together can result in a considerably more efficient, reliable and/or personalized processes and substances applicable.

In the automated cell processing, various measurement procedures are implemented. These serve to record the amount of tissue removed and to quantify and measure the quality of the final cell product before they are used. This will make it possible to conduct prospective studies within the scope of to make clinical application significantly easier. No more “external” measurements have to be carried out in the laboratory. The measurement can also be performed without interrupting the sterile, closed process. Transportation and decanting for measurement are no longer necessary.

The quantification procedures and quality measurement methods result from the following technical implementations:
Impedance measurement (for measuring the cell number or cell quality of the end product and/or an intermediate product);
Optical measurement (for measuring the cell flow measurement (volume flow or mass flow measurement to measure the amount of educt for a specific process step); and/or
Cellularity measurement with antibodies (Iden).

AMFAT (autologous micro-fragmented adipose tissue) process with/without enzyme digestion; quantity and quality of the tissue are measured with different measuring methods. The initial tissue is measured by impedance, DC resistance and sound propagation velocity measurements to quantify the amount of tissue applied to the machine. If enzymatic digestion is required in the AMFAT process, the correct amount of enzyme can be applied for digestion (1st process). The cell quality according to the AMFAT process (1st process) is automatically measured by optical density measurement.

Mechanical SVF with/without enzyme digestion; quantity and quality of tissue are measured by optical density measurement, colorimetric and fluorescence methods.

Additional volume and viscosity measurement can be made for optimum application viscosity after each operation. Through the implemented measurement procedures, a feedback process is created and programmed which offers the following process advantages:

After determining the quantity of fat introduced, the required quantity of enzyme is determined and the exact dose is added in the following process steps.

A small sample of the tissue to be processed is first sent through the process with different measuring steps in order to optimally determine the process parameters for the processing of the tissue to be processed by the results of the sample. (Some measuring steps can only be carried out with a small sample for technical and economic reasons).

After determining the amount of fat tissue introduced, the amplitude of the “shaking” process is adjusted individually in order to keep the mechanical forces as low as possible and not to endanger the cell quality.

After the “shaking” process a quality and quantity measurement is also carried out, which means that the subsequent centrifugation is also adjusted to the quality and quantity of the first “shaking” product with an automatic centrifugation speed control.

Interaction of 3 process steps via feedback (swivel+centrifuge+enzymatic processing) to a “user-specific” optimized treatment solution for clinical use. AMFAT (swivel), centrifugation (mechanical SVF), enzymatic digestion (enzymatic SVF) are combined in one system. In this system an optimized “cell” result can be achieved by a “feedback” system through computer programming. Therefore, different quantity and quality tracking systems are used to obtain a “feedback” loop that optimizes the mechanical and enzymatic cell processing steps.

The “Feedback” tracker enables the user to optimize the cell quality for each use. This is done by different quantification steps, which enter the original tissue. After the quantification of the tissue, a “probe” of approx. 50 ml goes into the individually programmed test cycle (AMFAT, mechanical SVF, enzymatic SVF). The terms AMFAT (autologous micro fragmented adipose tissue) or mechanic or enzymatic SVF (stromal vascular fraction) is sometimes also designated as “Millifat”, “Microfat” and “Nanofat”.

By generating “Super SVF” with two enzymatic processing (shaking and centrifugation process) the mechanical damage to the cells is reduced by the reduced amplitude of the shaker and centrifuge, resulting in a higher quality enzymatic SVF.

The term “component” is intended to comprise a unitary entity and/or one or more entities that are distributed in location. Thus, a component can comprise an integral element as well as an assembly of same and/or different elements.

Machine learning (ML) comprises computer algorithms that improve automatically through experience and by the use of data. It is seen as a part of artificial intelligence. Machine learning algorithms or approaches build a model based on sample data, known as “training data”, in order to make predictions or decisions without being explicitly programmed to do so. The term “training” is intended to comprise this kind of machine learning approach.

Supervised learning (SL) is the machine learning task of learning a function that maps an input to an output based on example input-output pairs. It infers a function from labeled training data consisting of a set of training examples. In supervised learning, each example is a pair consisting of an input object (typically a vector) and a desired output value (also called the supervisory signal). A supervised learning algorithm analyzes the training data and produces an inferred function, which can be used for mapping new examples.

Unsupervised learning (UL) is a type of algorithm that learns patterns from untagged data. The machine is forced to build a compact internal representation of its world and then generate imaginative content. In contrast to supervised learning (SL) where data is tagged by a human, e.g., as “car” or “fish” etc., UL exhibits self-organization that captures patterns as neuronal predilections or probability densities. The other levels in the supervision spectrum are reinforcement learning where the machine is given only a numerical performance score as its guidance, and semi-supervised learning where a smaller portion of the data is tagged. Two broad methods in UL are Neural Networks and Probabilistic Methods.

Deep learning (also known as deep structured learning) is part of a broader family of machine learning methods based on artificial neural networks with representation learning. Learning can be supervised, semi-supervised or unsupervised.

Deep-learning architectures such as deep neural networks, deep belief networks, graph neural networks, recurrent neural networks and convolutional neural networks have been applied to fields including computer vision, speech recognition, natural language processing, machine translation, bioinformatics, drug design, medical image analysis, material inspection and board game programs, where they have produced results comparable to and in some cases surpassing human expert performance.

The adjective “deep” in deep learning refers to the use of multiple layers in the network.

Explainable AI (XAI) or explainable artificial intelligence can be applied and is understood as artificial intelligence (AI) in which the results of the solution can be understood by humans.

The before definitions have been taken from Wikipedia on Jun. 22, 2021. These are herein incorporated by reference including further information contained therein.

Below, system embodiments will be discussed. These embodiments are abbreviated by the letter “S” followed by a number. Whenever reference is herein made to “system embodiments”, these embodiments are meant.
S1. System for tissue derived cell processing comprising:
   a. a controlling component (41); and
   b. a driving and/or heating component (50) configured to host a cartridge (60) and to drive and/or heat the cartridge (60).
S2. System according to the preceding system embodiment further comprising a cartridge (60) comprising a shaker and/or a centrifuge.
S3. System according to the preceding system embodiment wherein the cartridge (60) can be connected by and driven and/or heated by the driving and/or heating component (50).
S4. System according to any of the preceding system embodiments wherein the controlling component (41) and the driving and/or heating component (50) are modular and connected to each other so that the controlling component can control the driving and/or heating component (50).
S5. System according to any of the preceding system embodiments wherein the controlling component (41) comprises at least one slot (44-47) for the insertion of a reservoir for a substance.
S6. System according to any of the preceding system embodiments wherein the controlling component (41) comprises at least one slot (44-47) for the insertion of a syringe of a substance and wherein the controlling component (41) comprises an expelling component for expelling the content of the syringe(s) in controlled fashion.
S7. System according to the preceding system embodiment wherein the expelling component comprises one or more activator(s) for a/each plunger of the syringe(s) wherein each activator is able to push and/or retract the plunger of the syringe(s).
S8. System according to any of the preceding system embodiments wherein the controlling component (41) and the driving and/or heating component (50) are configured to be connected to each other with a liquid connection in order to enable any substance to be transported to and/or through the driving and/or heating component (50).
S9. System according to any of the preceding system embodiments wherein the driving and/or heating component (50) and the cartridge (60) are configured to be connected to each other with a liquid connection in order to enable any substance to be transported to and/or through the driving and/or heating component (50) into and/or out of the cartridge.
S10. System according to any of the preceding system embodiments further comprising one or more valves (71) configured to enable a flow of fluids from the controlling component (41) to the cartridge and/or vice versa.
S11. System according to any of the preceding system embodiments further comprising one or more valves (71) configured to enable a flow of fluids from the controlling component (41) to the cartridge and/or vice versa.
S12. System according to any of the preceding system embodiments further comprising one or more valves (71) configured to enable a flow of fluids from the controlling component (41) to the cartridge and/or vice versa through the driving and/or heating component (50).
S13. System according to any of the preceding system embodiments wherein the controlling component (41) and the driving and/or heating element (50) are configured to be electrically and/or fluidly connected.
S14. System according to any of the preceding system embodiments wherein the controlling component (41) and the driving and/or heating element (50) are configured for a snap-in connection.
S15. System according to any of the preceding system embodiments wherein the controlling component (41) and the driving and/or heating element (50) are configured for a connection verification upon a proper connection.
S16. System according to any of the preceding system embodiments wherein the controlling component (41) and the driving and/or heating element (50) are configured for a snap-in connection with a connection locking.
S17. System according to any of the preceding system embodiments wherein the driving and/or heating element (50) and the cartridge (60) are configured to be electrically and/or fluidly connected.
S18. System according to any of the preceding system embodiments wherein the driving and/or heating element (50) and the cartridge (60) are configured for a snap-in connection.
S19. System according to any of the preceding system embodiments wherein the driving and/or heating element (50) and the cartridge (60) are configured for a connection verification upon a proper connection.
S20. System according to any of the preceding system embodiments wherein the driving and/or heating element (50) has a recess (51) and the cartridge (60) a male member for a connection between both.
S21. System according to any of the preceding system embodiments wherein the controlling component (41) comprises a user interface (42), such as a GUI.
S22. System according to any of the preceding system embodiments wherein the controlling component (41) comprises a surface with a user interface (42) and/or syringe slots (44-47).
S23. System according to the preceding system embodiment wherein the surface is sloped towards the front.
S24. System according to any of the preceding system embodiments wherein the controlling component (41) is configured to host a container for cell enriched substance.
S25. System according to any of the preceding system embodiments wherein the controlling component (41) is configured to host a system to be filled with cell enriched substance.
S26. System according to any of the preceding system embodiments wherein the controlling component (41) comprises a product recess (43) for a container with cell enriched substance.
S27. System according to any of the preceding system embodiments wherein the controlling component (41) comprises a product recess (43) for a syringe with cell enriched substance.
S28. System according to any of the preceding system further comprising at least one sensor (72,73) for sensing the presence and/or composition and/or a physical parameter of substance in the system.
S29. System according to any of the preceding system embodiments wherein the system comprises an analysis component (75) that is configured to provide at least one marker and/or antigen marker of the following kind:
   a. CD271+
   b. CD146+
   c. CD166+
   d. CD13+
   e. CD166
   f. CD105
   g. CD90 and/or
   h. CD271.
S30. System according to any of the preceding system further comprising an AMFAT component (11).
S31. System according to any the preceding embodiments wherein the system comprises a centrifuge component (60).
S32. System according to any of the preceding system embodiments further comprising an inlet for feeding tissue into the AMFAT component.
S33. System according to any of the preceding system embodiments further comprising a cell quantifying and/or qualifying component.
S34. System according to the preceding embodiment wherein the cell quantifying and/or qualifying component comprises an impedance sensor.
S35. System according to any of the preceding embodiments wherein the impedance sensor is configured to quantify the volume flow in the system.
S36. System according to any of the preceding embodiments wherein the system comprises an optical sensor.
S37. System according to any of the preceding embodiments wherein the cell quantifying and/or qualifying component comprises the optical sensor.
S38. System according to any of the preceding embodiments wherein the optical sensor is configured to quantify the volume, mass and/or density flow in the system.
S39. System according to any of the preceding embodiments wherein the system comprises an immunoassay unit that is configured to quantify antigens present in the volume flow in the system.
S40. System according to any of the preceding embodiments wherein the system comprises a colorimetric system or fluorescence measurement unit that is configured to quantify antigens present in the volume flow in the system.
S41. System according to any of the preceding embodiments wherein the system further comprises at least one resistance sensor that is configured to quantify the cells in the volume flow in the system.
S42. System according to any of the preceding embodiments wherein the system comprises a sound source that us configured to send a sound signal to quantify the volume flow.
S43. System according to any of the preceding embodiments wherein the cell quantifying and/or qualifying component is configured to generate cell-relevant data at least based on the quantification of the volume flow in the system.
S44. System according to any of the preceding embodiments wherein the AMFAT component comprises a computing unit configured to access cell-relevant data from the cell quantifying and/or qualifying component.
S45. System according to any of the preceding embodiments wherein the cell-relevant data comprises at least one of:
   i. quantity;
   ii. quality;
   iii. vesicles;
   iv. exosomes/surface proteins;
   v. temperature;
   vi. enzyme data;
   vii. type of cell;
   viii. additive data;
   ix. viscosity data;
   x. composition data; and
   xi. size data.
S46. System according to any of the preceding embodiments wherein the AMFAT component and the cell quantifying and/or qualifying component are integrated on a single device.
S47. System according to any of the preceding embodiments wherein the AMFAT component is configured to automatically generate a swivel amplitude based on cell-relevant data.
S48. System according to any of the preceding embodiments wherein the AMFAT component further comprises an enzyme inlet.
S49. System according to any of the preceding embodiments wherein an enzyme component is configured to extract enzyme, preferably via enzyme inlet.
S50. System according to any of the preceding embodiments wherein an enzyme component is configured to extract enzyme, preferably via enzyme inlet, preferably based on cell-relevant data.
S51. System according to any of the preceding embodiments wherein the system comprises a syringe driver.
S52. System according to any of the preceding embodiments wherein the syringe driver is configured to extract the tissue.
S53. System according to any of the preceding embodiments wherein the syringe driver is configured to measure viscosity of sample, preferably using force measurement.
S54. System according to any of the preceding embodiments wherein the syringe driver is configured to measure volume of sample.
S55. System according to any of the preceding embodiments wherein the syringe driver (10) is configured to input the adipose tissue in the AMFAT component.
S56. System according to any of the preceding embodiments wherein the AMFAT component is configured to automatically generate a swivel time based on cell-relevant data.
S57. System according to any of the preceding embodiments wherein the system is further configured to input the swivel amplitude and/or swivel time data to a swivel component.
S58. System according to any of the preceding embodiments wherein the swivel component comprises a weight measuring component.
S59. System according to the preceding embodiments wherein the AMFAT component is equipped with the swivel component.
S60. System according to any of the preceding embodiments wherein the AMFAT component comprises the cell quantifying and/or qualifying component.
S61. System according to any of the preceding embodiments wherein the AMFAT component is configured to generate at least a portion of cell-relevant data.
S62. System according to any of the preceding embodiments wherein the AMFAT component is configured to generate at least a portion of cell-relevant data after the swiveling.
S63. System according to any of the preceding embodiments wherein the AMFAT component is configured to input the volume flow to the cell processing component to generate at least a portion of cell-relevant data.
S64. System according to any of the preceding embodiments wherein the AMFAT component is further equipped with a temperature controller.
S65. System according to any of the preceding embodiments wherein the temperature controller is configured to maintain an optimized temperature of the volume flow in the system.
S66. System according to any of the preceding embodiments wherein the optimized temperature is automatically determined by the cell quantifying and/or qualifying component, preferably based on cell-relevant data.
S67. System according to any of the preceding embodiments wherein the system comprises an additive component.
S68. System according to any of the preceding embodiments wherein the additive component is configured to input at least one determined additive to the volume flow in a system, preferably in the system outlet.
S69. System according to any of the preceding embodiments wherein the system further comprises a saline component configured to control the salinity of the volume flow in the system.
S70. System according to any of the preceding embodiments wherein the system further comprises a centrifugation component (66).
S71. System according to any of the preceding embodiments wherein the centrifugation component (66) comprises a weight measuring component.
S72. System according to any of the preceding embodiments wherein the temperature controller is configured to maintain the optimized temperature of the volume in the centrifuge component (66).
S73. System according to any of the preceding embodiments wherein the AMFAT component and the centrifugation component are configured to be integrated on a single device.
S74. System according to any of the preceding embodiments wherein the centrifugation component (66) comprises at least one of enzyme inlet and volume inlet.
S75. System according to any of the preceding embodiments wherein the centrifugation component (66) comprises an outlet (62).
S76. System according to any of the preceding embodiments wherein the centrifugation data is generated based on at least one portion of cell-relevant data.
S77. System according to the preceding embodiment wherein the centrifugation data comprises at least one of:
   i. centrifugation speed, and
   ii. centrifugation time, and
   iii. temperature of the centrifuge, and
   iv. number of centrifugation cycles.
S78. System according to any of the preceding embodiments wherein the system comprises a user interface.
S79. System according to any of the preceding embodiments wherein the system is configured to extract user data.
S80. System according to any of the preceding embodiments wherein the user interface is configured to extract the user data.
S81. System according to any of the preceding embodiments wherein the cell-relevant data is associated with the user data.
S82. System according to any of the preceding embodiments wherein the system is configured to store at least a portion of user data on a server.
S83. System according to any of the preceding embodiments wherein the system is further configured to store cell-relevant data on the server.
S84. System according to any of the preceding embodiments wherein the system is configured to determine the cell quality on the basis of the quantity of undifferentiated stem cells and/or the quantity of differentiated stem cells and/or the ratio of undifferentiated stem cells versus differentiated stem cells present in the tissue.
S85. System according to any of the preceding embodiments wherein the system further comprises a qualifying component that is configured to determine the quality of the tissue based on a cellularity measurement with antibodies, optically and/or with fluidic and/or microfluidic.
S86. System for tissue derived cell processing comprising sensors (72,73,81a) for sensing the physical and/or mechanical properties of substance(s), lipoaspirate, substances, catalysts, enzymes and/or components (41,50,60) and actuators (50,63,64,66,71,83,84) for controlling the cell processing.
S87. System according the preceding system embodiment and any other preceding system embodiments.
S88. System according to any of the 2 preceding embodiments wherein the system is configured to obtain also treatment relevant data.
S89. System according to any of the 3 preceding system embodiments wherein the system is configured to obtain and/or process health-related data of a patient.
S90. System according to any of the 4 preceding system embodiments wherein the system is configured to control the actuators (50,63,64,66,71,83,84) on the basis of sensor-based sensor data and/or the health-related data.
S91. System according to the preceding system embodiment further comprising software that is configured to assist in controlling the actuators (50,63,64,66,71,83,84).
S92. System according to the preceding system embodiment wherein the system is configured to train the software.
S93. System according to any of the 2 preceding system embodiments wherein the system is configured to train the software on the basis of supervised learning.
S94. System according to any of the 3 preceding system embodiments wherein the system is configured to train the software on the basis of deep learning.
S95. System according to any of the 4 preceding system embodiments wherein the system is configured to train the software on the basis of explainable artificial intelligence (XAI).
S96. System according to any of the 5 preceding system embodiments wherein the system comprises software that has been trained.

Below, cartridge embodiments will be discussed. These embodiments are abbreviated by the letter “C” followed by a number. Whenever reference is herein made to “cartridge embodiments”, these embodiments are meant.
C1. Cartridge configured to be used in a system according to any of the preceding system embodiments.
C2. Cartridge according to the preceding cartridge embodiment comprising a shaker (63) and/or a centrifuge (66) for shaking and/or centrifuging lipoaspirate.
C3. Cartridge according to any of the preceding cartridge embodiments wherein a shaker (63) and a centrifuge (66) are hosted in the cartridge.
C4. Cartridge according to the preceding cartridge embodiment wherein the shaker a sieve (64) is locate in the cartridge.
C5. Cartridge according to the preceding cartridge embodiment wherein the sieve (64) comprises one or more variable mesh size.
C6. Cartridge according to any of the preceding cartridge embodiments wherein a variable mesh size in the sieve (64) is realized by at least two mesh layers (64a,64b) that can be moved to each other.
C7. Cartridge according to any of the preceding cartridge embodiments wherein a variable mesh size in the sieve (64) is realized by at least two mesh layers (64a,64b) that can be rotated to each other.
C8. Cartridge according to any of the preceding cartridge embodiments comprising an input (61) and/or an output (62) that are configured to be fluidly connected to another component, such as a driving and/or heating component and/or a controlling component (41).
C9. Cartridge according to the preceding cartridge embodiment wherein the output (62) comprises an outlet for a cell-enriched substance and a second outlet for a cell-impoverished substance.
C10. Cartridge according to the preceding cartridge embodiment wherein the first and second outlets are concentrically arranged.
C11. Cartridge according to the preceding cartridge embodiment wherein the first and second outlets are concentrically arranged with the outlet for the cell-enriched substance being located exterior to the second outlet for the cell-impoverished substance.
C12. Cartridge according to any of the preceding cartridge embodiments wherein the shaker (63) is configured to also shake the centrifuge (66).
S85. System with a cartridge according to any of the preceding cartridge embodiments.

Below, method embodiments will be discussed. These embodiments are abbreviated by the letter “M” followed by a number. Whenever reference is herein made to “method embodiments”, these embodiments are meant.
M1. Method for tissue derived cell processing, particularly with a system according to any of the preceding system embodiments, comprising the steps of controlling by a controlling component (41) and driving and/or heating a cartridge (60) by a driving and/or heating component (50) configured to host the cartridge (60).
M2. Method according to the preceding method embodiment further comprising a cartridge (60) comprising a shaker and/or a centrifuge.
M3. Method according to the preceding method embodiment wherein the cartridge (60) can be connected by and driven and/or heated by the driving and/or heating component (50).
M4. Method according to any of the preceding method embodiments wherein the controlling component (41) and the driving and/or heating component (50) are modular and connected to each other so that the controlling component can control the driving and/or heating component (50).
M5. Method according to any of the preceding method embodiments wherein the controlling component (41) comprises at least one slot (44-47) for the insertion of a reservoir for a substance.
M6. Method according to any of the preceding method embodiments wherein the controlling component (41) comprises at least one slot (44-47) for the insertion of a syringe of a substance and wherein the controlling component (41) comprises an expelling component for expelling the content of the syringe(s) in controlled fashion.
M7. Method according to the preceding method embodiment wherein the expelling component comprises one or more activator(s) for a/each plunger of the syringe(s) wherein each activator is able to push and/or retract the plunger of the syringe(s).
M8. Method according to any of the preceding method embodiments wherein the controlling component (41) and the driving and/or heating component (50) are configured to be connected to each other with a liquid connection in order to enable any substance to be transported to and/or through the driving and/or heating component (50).
M9. Method according to any of the preceding method embodiments wherein the driving and/or heating component (50) and the cartridge (60) are configured to be connected to each other with a liquid connection in order to enable any substance to be transported to and/or through the driving and/or heating component (50) into and/or out of the cartridge.
M10. Method according to any of the preceding method embodiments further comprising one or more valves (71) configured to enable a flow of fluids from the controlling component (41) to the cartridge and/or vice versa.
M11. Method according to any of the preceding method embodiments further comprising one or more valves (71) configured to enable a flow of fluids from the controlling component (41) to the cartridge and/or vice versa.
M12. Method according to any of the preceding method embodiments further comprising one or more valves (71) configured to enable a flow of fluids from the controlling component (41) to the cartridge and/or vice versa through the driving and/or heating component (50).
M13. Method according to any of the preceding method embodiments wherein the controlling component (41) and the driving and/or heating element (50) are configured to be electrically and/or fluidly connected.
M14. Method according to any of the preceding method embodiments wherein the controlling component (41) and the driving and/or heating element (50) are configured for a snap-in connection.
M15. Method according to any of the preceding method embodiments wherein the controlling component (41) and the driving and/or heating element (50) are configured for a connection verification upon a proper connection.
M16. Method according to any of the preceding method embodiments wherein the controlling component (41) and the driving and/or heating element (50) are configured for a snap-in connection with a connection locking.
M17. Method according to any of the preceding method embodiments wherein the driving and/or heating element (50) and the cartridge (60) are configured to be electrically and/or fluidly connected.
M18. Method according to any of the preceding method embodiments wherein the driving and/or heating element (50) and the cartridge (60) are configured for a snap-in connection.
M19. Method according to any of the preceding method embodiments wherein the driving and/or heating element (50) and the cartridge (60) are configured for a connection verification upon a proper connection.
M20. Method according to any of the preceding method embodiments wherein the driving and/or heating element (50) has a recess (51) and the cartridge (60) a male member for a connection between both.
M21. Method according to any of the preceding method embodiments wherein the controlling component (41) comprises a user interface (42), such as a GUI.
M22. Method according to any of the preceding method embodiments wherein the controlling component (41) comprises a surface with a user interface (42) and/or syringe slots (44-47).
M23. Method according to the preceding method embodiment wherein the surface is sloped towards the front.
M24. Method according to any of the preceding method embodiments wherein the controlling component (41) is configured to host a container for cell enriched substance.
M25. Method according to any of the preceding method embodiments wherein the controlling component (41) is configured to host a method to be filled with cell enriched substance.
M26. Method according to any of the preceding method embodiments wherein the controlling component (41) comprises a product recess (43) for a container with cell enriched substance.
M27. Method according to any of the preceding method embodiments wherein the controlling component (41) comprises a product recess (43) for a syringe with cell enriched substance.
M28. Method according to any of the preceding method further comprising at least one sensor (72,73) for sensing the presence and/or composition and/or a physical parameter of substance in the method.
M29. Method according to any of the preceding method embodiments with the step of using at least one of the following marker and/or antigen marker of the following kind for an analysis:
   a. CD271+
   b. CD146+
   c. CD166+
   d. CD13+
   e. CD166
   f. CD105
   g. CD90 and/or
   h. CD271.
M30.
M31. Method for cell processing comprises automatically analyzing tissue to generate at least a portion of cell-relevant data.
M32. Method for cell processing comprises automatically analyzing a volume flow to generate at least a portion of cell-relevant data.
M33. Method according to any of the preceding embodiments comprises determining at least one operation parameter, at least based on cell-relevant data.
M34. Method according to any of the preceding embodiments wherein the step of analyzing tissue comprises measuring at least one of volume and weight and density of tissue.
M35. Method according to any of the preceding embodiments wherein the step of analyzing tissue further comprises measuring viscosity of the tissue.
M36. Method according to any of the preceding embodiments wherein the step of analyzing tissue further comprises measuring the number of cells in the tissue.
M37. Method according to any of the preceding embodiments wherein the step of analyzing tissue comprises measure the vitality of derived cells in the tissue.
M38. Method according to any of the preceding embodiments with the further step of determining the quality of the tissue based on a cellularity measurement with antibodies, optically and/or with fluidic and/or microfluidic.
M39. Method according to any of the preceding embodiments comprising the further step of determining the cell quality on the basis of the quantity of undifferentiated stem cells and/or the quantity of differentiated stem cells and/or the ratio of undifferentiated stem cells versus differentiated stem cells present in the tissue.
M40. Method according to any of the preceding embodiments comprising the further step of providing the undifferentiated stem cells adipose-derived and the differentiated stem cells cartilage or osteocyte-differentiated.
M41. Method according to any of the preceding embodiments wherein the operation parameter comprises at least one of:
   i. centrifuge speed; and
   ii. swivel amplitude; and
   iii. swivel time; and
   iv. swivel frequency; and
   V. centrifugation time; and
   vi. enzyme data; and
   vii. swivel temperature; and
   viii. centrifuge temperature; and
   ix. quantity of additive; and
   X. number of centrifugation cycles.
M42. Method for tissue derived cell processing comprising the steps of sensing the physical and/or mechanical properties of substance(s), lipoaspirate, substances, catalysts, enzymes and/or components (41,50,60) by sensors (72,73,81a) and controlling the cell processing by actuators (50,63,64,66,71,83,84).
M43. Method according to the preceding method embodiment and any other preceding method embodiments.
M44. Method according to any of the 2 preceding method embodiments wherein the method is configured to obtain also treatment relevant data.
M45. Method according to any of the 3 preceding method embodiments wherein the method is configured to obtain and/or process health-related data of a patient.
M46. Method according to any of the 4 preceding method embodiments wherein the method is configured to control the actuators (50,63,64,66,71,83,84) on the basis of sensor-based sensor data and/or the health-related data.
M47. Method according to any of the 3 preceding method embodiments wherein the method is configured to obtain and/or process health-related data of a patient.
M48. Method according to any of the 4 preceding embodiments wherein the method is configured to control the actuators (50,63,64,66,71,83,84) on the basis of sensor-based sensor data and/or the health-related data.
M49. Method according to the preceding method embodiment further with the step of controlling the actuators (50,63,64,66,71,83,84) by software.
M50. Method according to the preceding method embodiment wherein the method is configured to train the software.
M51. Method according to any of the 2 preceding method embodiments wherein the method is configured to train the software on the basis of supervised learning.
M52. Method according to any of the 3 preceding method embodiments wherein the method is configured to train the software on the basis of deep learning.
M53. Method according to any of the 4 preceding method embodiments wherein the method is configured to train the software on the basis of explainable artificial intelligence (XAI).
M54. Method according to any of the 5 preceding method embodiments wherein the method comprises applying software that has been trained.

Below, use embodiments will be discussed. These embodiments are abbreviated by the letter “U” followed by a number. Whenever reference is herein made to “use embodiments”, these embodiments are meant.
U1. Use of the system according to any of the preceding system embodiments for analyzing tissues.
U2. Use of the system according to any of the preceding system embodiments for carrying out the method according to any of the preceding method embodiments.
U3. Use of the system according to any of the preceding system embodiments for orthopedics, such as arthritis, tissue damage to cartilage.
U4. Use of the system according to any of the preceding embodiments for dermatology, such as chronic wounds, burns.
U5. Use of the system according to any of the preceding embodiments for restoration, reconstruction, or alteration, such as breast augmentation, dark circles.
U6. Use of the system according to any of the preceding embodiments for neurology, such as Alzheimer, Parkinson's.
U7. Use of the system according to any of the preceding embodiments for cardiology.
U8. Use of the system according to any of the preceding embodiments for internal medicine, such as to treat organ damage, for example liver damage.

Below, computer related product embodiments will be discussed. These embodiments are abbreviated by the letter “C” followed by a number. Whenever reference is herein made to “computer related product embodiments”, these embodiments are meant.
C1. A computer program product comprising instructions, which, when the program is executed by a user interface device, causes a user device to perform the method steps according to any method embodiment, which have to be executed on the user interface device, wherein the user interface device is according to any system embodiment that comprises a user device that is compatible to said method embodiment.
C2. A computer program product comprising instructions, which, when the program is executed by a combination of a server and user interface device, cause the server and the user interface device to perform the method steps according to any method embodiment, which have to be executed on the server and the user interface device, wherein the user device and the server is according to any system embodiment that comprises a sever and/or the user device that is compatible to said method embodiment.
C3. A computer program product comprising instructions, which, when the program is executed by a server, cause the server to perform the method steps according to any method embodiment, which have to be executed on the server, wherein the server is according to any system embodiment that comprises a server that is compatible to said method embodiment.
C4. A computer program product comprising instructions, which, when the program is executed by a cell quantifying and/or qualifying component, cause the cell quantifying and/or qualifying component to perform the method steps according to any method embodiment, which have to be executed on the cell quantifying and/or qualifying component, wherein the cell quantifying and/or qualifying component is according to any system embodiment that comprises a cell quantifying and/or qualifying component that is compatible to said method embodiment.
C5. A computer related product with a program that is configured for carrying out the method according to any of the preceding method embodiments.
C6. A computer related product with a program that is trained for carrying out the method according to any of the preceding method embodiments.
C7. A computer related product with a program that has been trained for carrying out the method according to any of the preceding method embodiments.

Below, diagnostic kit embodiments will be discussed. These embodiments are abbreviated by the letter “D” followed by a number. Whenever reference is herein made to “diagnostic embodiments”, these embodiments are meant.
D1. A diagnostic kit comprising a sterile package comprising any component according to any of the system embodiments.
D2. The diagnostic kit according to the preceding embodiment comprises a disposable cartridge (60).
D3. The diagnostic kit according to any of the preceding embodiments configured to be attached to the system according to any of the system embodiments.
D4. The diagnostic kit according to any of the preceding embodiments further comprises a saline component.
D5. The diagnostic kit according to any of the preceding embodiments comprises a swivel component, wherein the swivel component is configured to extract at least one of adipose tissue and enzyme and saline solution.
D6. The diagnostic kit according to any of the preceding embodiments comprises a centrifuge component, wherein the centrifuge component is configured to extract at least one of adipose tissue and enzyme and saline solution.
D7. The diagnostic kit according to any of the preceding embodiments comprises a catalyst component wherein the catalyst component is configured to input at least one catalyst in the output component.
D8. The diagnostic kit according to any of the preceding embodiments comprises the cell quantifying and/or qualifying component configured to send cell-relevant data to the swivel component.
D9. The diagnostic kit according to any of the preceding embodiments comprises the cell quantifying and/or qualifying component configured to send cell-relevant data to the centrifuge component.
D10. The diagnostic kit according to any of the preceding embodiments wherein the swivel component is configured to exchange volume flow with the centrifuge component.

Below, therapeutic embodiments will be discussed. These embodiments are abbreviated by the letter “T” followed by a number. Whenever reference is herein made to “therapeutic embodiments”, these embodiments are meant.
T1. Method of a therapeutic treatment of a patient by a method according to any of the preceding method embodiments.
T2. Method of a therapeutic treatment of a patient by a system according to any of the preceding system embodiments.
T3. Method of a therapeutic treatment for orthopedics, such as of arthritis, tissue damage to cartilage by a method according to any of the preceding method embodiments.
T4. Method of a therapeutic treatment for orthopedics, such as of arthritis, tissue damage to cartilage by a system according to any of the preceding system embodiments.
T5. Method of a therapeutic treatment for dermatology, such as of chronic wounds, burns by a method according to any of the preceding method embodiments.
T6. Method of a therapeutic treatment for dermatology, such as chronic wounds, burns by a system according to any of the preceding system embodiments.
T7. Method of a therapeutic treatment for restoration, reconstruction, or alteration, such as of breast augmentation, dark circles by a method according to any of the preceding method embodiments.
T8. Method of a therapeutic treatment for restoration, reconstruction, or alteration, such as of breast augmentation, dark circles by a system according to any of the preceding system embodiments.
T9. Method of a therapeutic treatment for neurology, such as of Alzheimer, Parkinson's by a method according to any of the preceding method embodiments.
T10. Method of a therapeutic treatment for neurology, such as of Alzheimer, Parkinson's by a system according to any of the preceding system embodiments.
T11. Method of a therapeutic treatment for cardiology and/or internal medicine, such as to treat organ damage, for example liver damage by a method according to any of the preceding method embodiments.
T12. Method of a therapeutic treatment for cardiology and/or internal medicine, such as to treat organ damage, for example liver damage by a system according to any of the preceding system embodiments.

The present invention will now be described with reference to the accompanying drawings, which illustrate embodiments of the invention. These embodiments should only exemplify, but not limit, the present invention.

### FIGURE DESCRIPTION

FIG. 1 schematically exemplifies a system hardware architecture in accordance with the present invention;

FIG. 2 schematically exemplifies a modular system according to an embodiment of the present invention.

FIG. 3 shows an exemplary setup according to an embodiment of the present invention.

FIG. 4 shows an exemplary combination of a shaker and centrifuge.

FIG. 5 depicts an example of an embodiment of a sieve in accordance with the present invention.

### DESCRIPTION OF PREFERRED EMBODIMENTS AS EXEMPLIFIED IN THE FIGURES

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

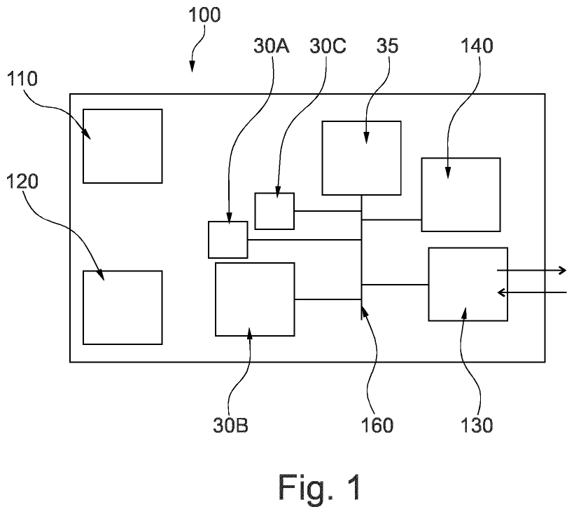

FIG. 1 provides a schematic of a computing device 100. The computing device 100 may comprise a computing unit 35, a first data storage unit 30A, a second data storage unit 30B and a third data storage unit 30C.

The computing device 100 can be a single computing device or an assembly of computing devices. The computing device 100 can be locally arranged or remotely, such as a cloud solution.

On the different data storage units 30 the different data can be stored, such as the operational parameters data on the first data storage 30A, the user data and/or cell relevant data and/or temperature data on the second data storage 30B and privacy sensitive data, such as the connection of the before-mentioned data to an individual, on the thirds data storage 30C.

Additional data storage can be also provided and/or the ones mentioned before can be combined at least in part. Another data storage (not shown) can comprise data specifying the composition or tissue and/or cell relevant data, such as volume, weight, viscosity between the different components etc. This data can also be provided on one or more of the before-mentioned data storages.

The computing unit 35 can access the first data storage unit 30A, the second data storage unit 30B and the third data storage unit 30C through the internal communication channel 160, which can comprise a bus connection 160.

The computing unit 30 may be single processor or a plurality of processors, and may be, but not limited to, a CPU (central processing unit), GPU (graphical processing unit), DSP (digital signal processor), APU (accelerator processing unit), ASIC (application-specific integrated circuit), ASIP (application-specific instruction-set processor) or FPGA (field programable gate array). The first data storage unit 30A may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The second data storage unit 30B may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM). The third data storage unit 30C may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

It should be understood that generally, the first data storage unit 30A (also referred to as encryption key storage unit 30A), the second data storage unit 30B (also referred to as data share storage unit 30B), and the third data storage unit 30C (also referred to as decryption key storage unit 30C) can also be part of the same memory. That is, only one general data storage unit 30 per device may be provided, which may be configured to store the respective encryption key (such that the section of the data storage unit 30 storing the encryption key may be the encryption key storage unit 30A), the respective data element share (such that the section of the data storage unit 30 storing the data element share may be the data share storage unit 30B), and the respective decryption key (such that the section of the data storage unit 30 storing the decryption key may be the decryption key storage unit 30A).

In some embodiments, the third data storage unit 30C can be a secure memory device 30C, such as, a self-encrypted memory, hardware-based full disk encryption memory and the like which can automatically encrypt all of the stored data. The data can be decrypted from the memory component only upon successful authentication of the party requiring to access the third data storage unit 30C, wherein the party can be a user, computing device, processing unit and the like. In some embodiments, the third data storage unit 30C can only be connected to the computing unit 35 and the computing unit 35 can be configured to never output the data received from the third data storage unit 30C. This can ensure a secure storing and handling of the encryption key (i.e. private key) stored in the third data storage unit 30C.

In some embodiments, the second data storage unit 30B may not be provided but instead the computing device 100 can be configured to receive a corresponding encrypted share from the database 60. In some embodiments, the computing device 100 may comprise the second data storage unit 30B and can be configured to receive a corresponding encrypted share from the database 60.

The computing device 100 may comprise a further memory component 140 which may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM). The memory component 140 may also be connected with the other components of the computing device 100 (such as the computing component 35) through the internal communication channel 160.

Further the computing device 100 may comprise an external communication component 130. The external communication component 130 can be configured to facilitate sending and/or receiving data to/from an external device (e.g. backup device 10, recovery device 20, database 60). The external communication component 130 may comprise an antenna (e.g. WIFI antenna, NFC antenna, 2G/3G/4G/5G antenna and the like), USB port/plug, LAN port/plug, contact pads offering electrical connectivity and the like. The external communication component 130 can send and/or receive data based on a communication protocol which can comprise instructions for sending and/or receiving data. Said instructions can be stored in the memory component 140 and can be executed by the computing unit 35 and/or external communication component 130. The external communication component 130 can be connected to the internal communication component 160. Thus, data received by the external communication component 130 can be provided to the memory component 140, computing unit 35, first data storage unit 30A and/or second data storage unit 30B and/or third data storage unit 30C. Similarly, data stored on the memory component 140, first data storage unit 30A and/or second data storage unit 30B and/or third data storage unit 30C and/or data generated by the commuting unit 35 can be provided to the external communication component 130 for being transmitted to an external device.

In addition, the computing device 100 may comprise an input user interface 110 which can allow the user of the computing device 100 to provide at least one input (e.g. instruction) to the computing device 100. For example, the input user interface 110 may comprise a button, keyboard, trackpad, mouse, touchscreen, joystick and the like.

Additionally, still, the computing device 100 may comprise an output user interface 120 which can allow the computing device 100 to provide indications to the user. For example, the output user interface 110 may be a LED, a display, a speaker and the like.

The output and the input user interface 100 may also be connected through the internal communication component 160 with the internal component of the device 100.

The processor may be singular or plural, and may be, but not limited to, a CPU, GPU, DSP, APU, or FPGA. The memory may be singular or plural, and may be, but not limited to, being volatile or non-volatile, such an SDRAM, DRAM, SRAM, Flash Memory, MRAM, F-RAM, or P-RAM.

The data processing device can comprise means of data processing, such as, processor units, hardware accelerators and/or microcontrollers. The data processing device 20 can comprise memory components, such as, main memory (e.g. RAM), cache memory (e.g. SRAM) and/or secondary memory (e.g. HDD, SDD). The data processing device can comprise busses configured to facilitate data exchange between components of the data processing device, such as, the communication between the memory components and the processing components. The data processing device can comprise network interface cards that can be configured to connect the data processing device to a network, such as, to the Internet. The data processing device can comprise user interfaces, such as:
output user interface, such as:
   screens or monitors configured to display visual data (e.g. displaying graphical user interfaces of the questionnaire to the user),
   speakers configured to communicate audio data (e.g. playing audio data to the user),
input user interface, such as:
   camera configured to capture visual data (e.g. capturing images and/or videos of the user),
   microphone configured to capture audio data (e.g. recording audio from the user),
   keyboard configured to allow the insertion of text and/or other keyboard commands (e.g. allowing the user to enter text data and/or other keyboard commands by having the user type on the keyboard) and/or trackpad, mouse, touchscreen, joystick—configured to facilitate the navigation through different graphical user interfaces of the questionnaire.

The data processing device can be a processing unit configured to carry out instructions of a program. The data processing device can be a system-on-chip comprising processing units, memory components and busses. The data processing device can be a personal computer, a laptop, a pocket computer, a smartphone, a tablet computer. The data processing device can be a server, either local and/or remote. The data processing device can be a processing unit or a system-on-chip that can be interfaced with a personal computer, a laptop, a pocket computer, a smartphone, a tablet computer and/or user interface (such as the upper-mentioned user interfaces).

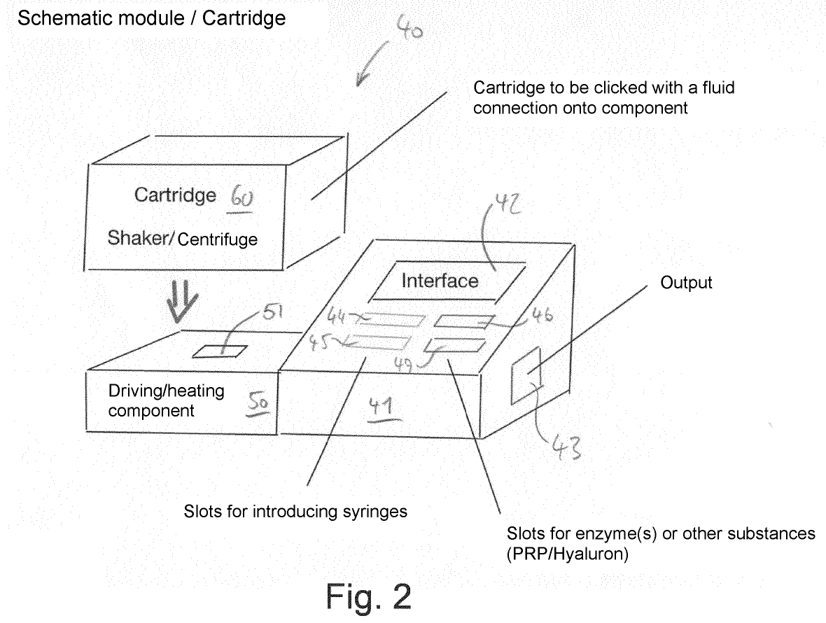

As can be seen in FIG. 2, the system according to the present invention can comprise an arrangement 40 with a number of modules for carrying out the cell processing in accordance with the present invention. The arrangement 40 can comprise a controlling component 41, a driving and/or heating component 50 and/or a cartridge 60 with a shaker and/or centrifuge.

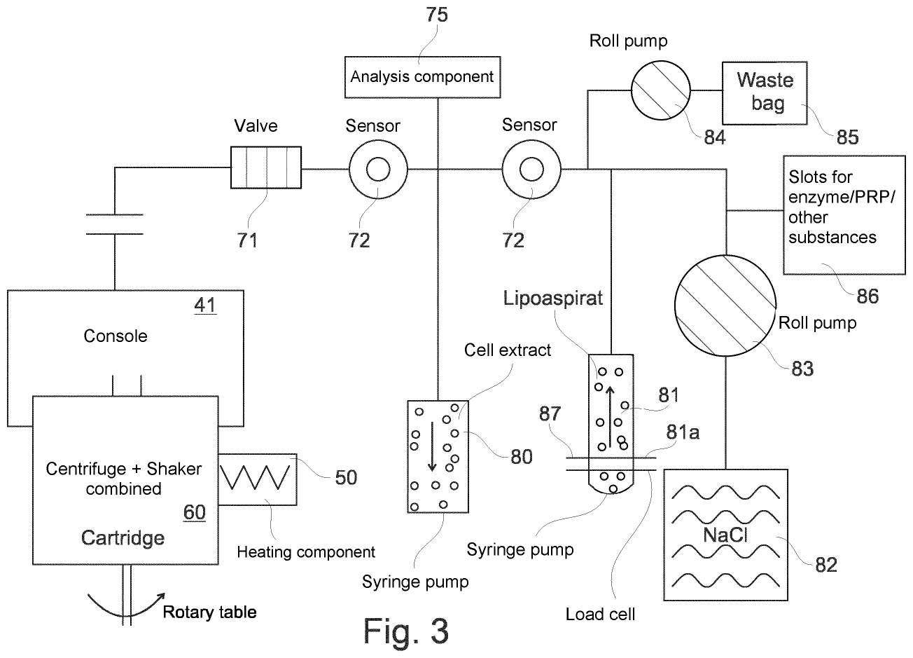

The controlling component 41 can have an interface 42, such as a GUI, keypad etc. It can also comprise slots 44-47 for introducing syringes containing different substances as will be described further below, particularly with respect to FIG. 3. There can also be an output 43 provided where the processed cells are dispelled.

Attached to the controlling component 41 is the driving and/or heating component 50. It can be formed integral or modular. A connector (not shown) can be provided to transmit signals between those components 41 and 50.

On top of the driving and/or heating component 50 the cartridge 60 can be attached. All those arrangements of component can vary, needless to say. The cartridge 60 can be clicked onto the driving and/or heating component 50 with a fluid connection being realized to the controlling component 41. A recess 51 can be provided in component 50 for allowing the entry of a respective male member of cartridge 60 for the fluid connection.

FIG. 3 shows just one out of a large number of possibilities to arrange different components and features in accordance with the invention. In the example shown a syringe or container with a lipoaspirate 81 is shown that can be weighed on a weighing cell 81a. There is also shown a reservoir of saline or NaCl 82 that can be delivered with a pump 83, such as a roll pump 83. There can be further slots 86 for one or more enzyme(s), platelet rich plasma (PRP) and/or other substances, such as catalysts etc. They can be provided by syringes, and the slot 86 can then provide one or more respective syringe slots with one or more expelling mechanism(s). There can be one or more sensor(s) 72, 73 in order to check the presence and/or composition of the material from those slots and reservoirs 81, 82, 86. A analysis component 75 can be connected to the sensors in order to analyze the amount, presence and/or composition of material in the line. The analysis component 75 can make use of markers such as: CD271+, CD146+, CD166+, CD13+, CD166, CD105, CD90 and/or CD271. A valve 71 can be arranged for allowing the composition to enter via a console or controlling component 41 a cartridge 60 that is hosting the centrifuge and shaker (not shown in detail). A heating component 50 can be affiliated. The respective driving of the centrifuge and/or shaker can be arranged in a combined driving and/or heating component 50 as shown in FIG. 2 or in separate components (not shown).

After the separation and/or extraction of the cells, those can be transferred via the valve 71 and at least one of the sensors 72 to a cell extract reservoir 80 that can be a syringe filled with the cell extract or cell suspension.

Also shown is a waste reservoir 85, such as a waste bag that is filled with the remaining composition from the lipoaspirate and potentially other substances besides the cell extract. A pump 84, such as a roll pump 85, can be configured to transfer the remaining composition into the waste reservoir 85.

As mentioned, many other configurations can be provided with less or more components.

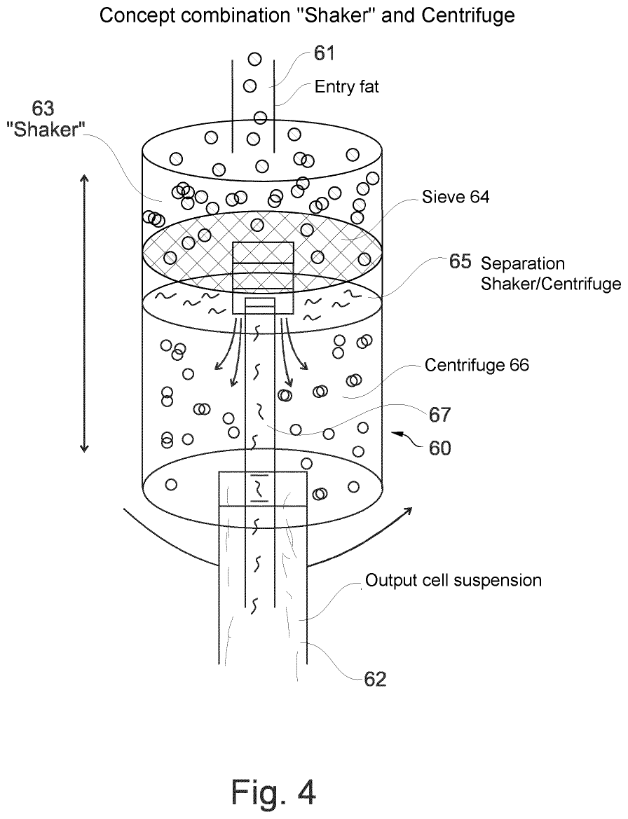

FIG. 4 is an example of a combined shaker and centrifuge cartridge 60. It can have an input of lipoaspirate with further substances. The cartridge 60 can host further components or sections that can also be provided separately. A shaker 63 is shaking the cartridge 60 or at least a section thereof in order to allow a proper mixing of compositions and/or substances. There is a sieve and/or filter 64 shown as one example through which the lipoaspirate and further substances are pressed or pulled. This sieve 64 is explained further below with respect to FIG. 4. The whole cartridge or at least parts thereof can be shaken by shaker 63.

After a first filtering or sieving the remaining material is transferred into the centrifuge where the cell suspension or cell extract is enriched and the remaining material is separated at least in considerable amounts or parts. This can be extracted through an output 62. There is also shown a central part of the output 62 that is transferring the remaining material apart from the cell suspension.

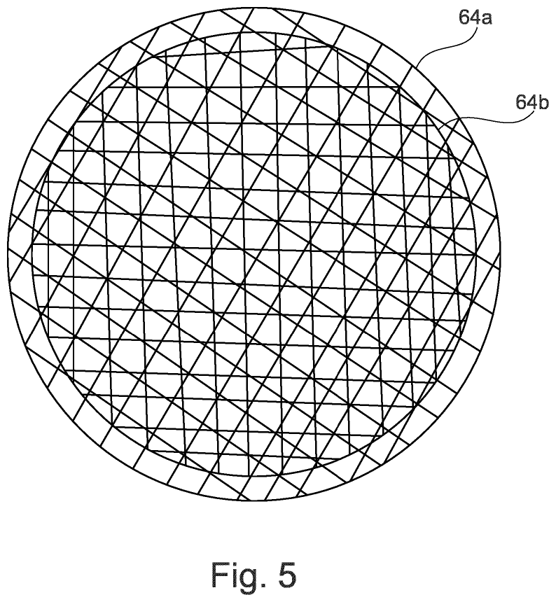

FIG. 5 exemplifies a filter or sieve 64 for a first filtering of the lipoaspirate and potentially further substances. In order to adapt the mesh size, the sieve can have two layers (64a, 64b) with geometrical pores that vary their size in case the two layers are mutually rotated to each other.

The system can comprise an AMFAT component. The AMFAT component 11 can include fat tissue washing process or shaking or swiveling. The amplitude for swiveling can be pre-determined using quantified cell-relevant data by the cell quantifying and/or qualifying component. Each module can have its own cell process component (not shown).

Further, the system can comprise one or more catalyst component(s). The catalyst component can be configured to add various products such as hyaluronic acid or Platelet Rich plasma (PRP). This admixture is beneficial for the effect of cell therapy through the added growth factors from the input sample (PRP) and positive for the longer retention in the application area through the hyaluronic acid.

Various processes from each module can be used and individually combined by separate components. For example, an AMFAT component can be used without the centrifuge component at first and later the centrifuge component can be added to the AMFAT component. The process steps can also be combined individually and performed simultaneously. Again, using each individual combination, the “feedback” process is possible at any time due to the implemented quantity and quality tracker and the user-friendly software application.

The different process steps can be carried out in an automated process, but are separated by a controlled fat quantity quantification (e.g. 450 ml in total, the first 300 ml for AMFAT, which is fed into the system, and the remaining 150 ml is converted into enzymatic SVF. This can facilitate a possibility to combine different process steps in an automated process at the same time (e.g. breast augmentation in combination with enzymatic SVF for a longer fat retention). This can thus save a lot of time and allows a parallel workflow.

Possibility of combination with current therapies (i.e. hyaluronic acid or PRP (autologous platelet-rich plasma, enzymes))—PRP can be added directly via the catalyst component—again separately at each process step. This means a variety of different processing options with additional substances.

The cell-relevant data can comprise volume, weight, density of the adipose derived tissue. The measurement can be done at an insertion point. The measurement can also be done in the syringe, preferably using the force measurement.

Based on the cell-relevant data a step of deciding the addition of a catalyst/enzyme is made. The measurement via antigens on the cell surface of the stem cell can also be made. The processing parameter can also be calculated based on cell-relevant data and can be adjusted based on user data. The processing parameter can also be adjusted based on a first output.

The lipoaspirate and/or enzyme/catalyst can be added to the volume flow via a syringe. The addition can take place after the quantity measurement and before the volume is transferred to the product syringe 80 as shown in FIG. 3.

A diagnosis kit can be attached to the system. The disposable diagnostic kit can consist a hard-plastic shaker/swivel device made of plastic, centrifuge, hoses, connectors, inlets, etc.

The diagnostic kit can comprise swivel component (shown in the figure as shaker) and the centrifuge component (shown in the figure as centrifuge). The entire can be delivered sterile. Access to the kit can be closed (e.g. with sterile Luer-Lock caps) until the kit is used. Storage of the kit for several months can be possible. The hoses can comprise an inner diameter of 3 to 6 mm, such as 4.8 mm and an outer diameter of 4 to 10 mm, such as 8 mm. The length and diameter of all hoses can be optimized to minimize the loss of the final product.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1-15. (canceled)

16. A system for tissue-derived cell processing comprising:
(a) a controlling component; and
(b) a driving and/or heating component configured to host a cartridge and to drive and/or heat the cartridge.

17. The system according to claim 16, further comprising a cartridge comprising a shaker and/or a centrifuge.

18. The system according to claim 17, wherein the cartridge can be connected by and driven and/or heated by the driving and/or heating component.

19. The system according to claim 16, wherein the controlling component and the driving and/or heating component are modular and connected to each other so that the controlling component can control the driving and/or heating component.

20. The system according to claim 16, wherein the controlling component comprises at least one slot for insertion of a reservoir for a substance.

21. The system according to claim 16, wherein the controlling component comprises at least one slot for insertion of a syringe of a substance and wherein the controlling component comprises an expelling component for expelling the content of the syringe(s) in controlled fashion.

22. The system according to claim 21, wherein the expelling component comprises one or more activator(s) for a/each plunger of the syringe(s) wherein each activator is able to push and/or retract the plunger of the syringe(s).

23. The system according to claim 16, wherein the controlling component and the driving and/or heating component are configured to be connected to each other with a liquid connection in order to enable any substance to be transported to and/or through the driving and/or heating component.

24. The system according to claim 16, wherein the driving and/or heating component and the cartridge are configured to be connected to each other with a liquid connection in order to enable any substance to be transported to and/or through the driving and/or heating component into and/or out of the cartridge.

25. The system according to claim 16 further comprising one or more valves configured to enable a flow of fluids from the controlling component to the cartridge and/or vice versa.

26. The system according to claim 16 further comprising one or more valves configured to enable a flow of fluids from the controlling component to the cartridge and/or vice versa through the driving and/or heating component.

27. The system according to claim 16, wherein the controlling component and the driving and/or heating element are configured to be electrically and/or fluidly connected.

28. A method for tissue-derived cell processing, particularly with a system according to claim 16, the method comprising the steps of controlling the process by controlling component driving and/or heating a cartridge by a driving and/or heating component configured to host the cartridge.

29. A computer-related product for carrying out the method according to claim 28.
